(19) United States
(12) Patent Application Publication
(10) Pub. No.: US 2023/0330356 A1
(43) Pub. Date: Oct. 19, 2023

(54) PRESSURIZED SUBLINGUAL DELIVERY DEVICE AND METHODS FOR MANUFACTURE AND USE OF THE SAME

(71) Applicant: Air 2 LLC, Centennial, CO (US)

(72) Inventors: Robert L. Senter, Centennial, CO (US); Robert E. Matuszewski, Centennial, CO (US)

(73) Assignee: Air 2 LLC, Centennial, CO (US)

(21) Appl. No.: 18/197,686

(22) Filed: May 15, 2023

Related U.S. Application Data
(63) Continuation-in-part of application No. 18/300,992, filed on Apr. 14, 2023.
(60) Provisional application No. 63/331,497, filed on Apr. 15, 2022.

(51) Int. Cl. A61M 11/00

(52) U.S. Cl. CPC A61M 11/001; A61M 2210/0643

(57) ABSTRACT

The present disclosure relates to an encapsulated, pressurized, sublingual nano mist vapor delivery device, which may deliver a formula, including a micellized liquid nicotine formula. The device comprises a protective receptacle or housing, which preferably houses at least one canister, and a receptacle cap. The device may also comprise a custom actuator in communication with a metered valve for delivering a precise, known volume of at least one micellized liquid nicotine formula. Methods of manufacture and use are also disclosed herein.

### CROSS-REFERENCE TO RELATED APPLICATIONS

The present application is a continuation-in-part of U.S. patent application Ser. No. 18/300,992 filed on Apr. 14, 2023, which in turn claims priority under 35 U.S.C. § 119(e) to U.S. Provisional Patent Application Ser. No. 63/331,497, filed Apr. 15, 2022. Each of the aforementioned applications is hereby incorporated by reference herein in their entireties.

### FIELD OF THE INVENTION

Aspects of the present disclosure relate generally to the field of medical devices, and more particularly an encapsulated, pressurized, sublingual nano mist vapor delivery device, which according to preferred embodiments contains a micellized liquid nicotine formula.

### BACKGROUND

The Center for Disease Control and Prevention (“CDC”) validates that smoking can cause cancer, most often lung cancer, and then block the human body from fighting the disease. It is well known in the art that the poisons in cigarette smoke can affectively weaken the body's immune system, creating a greater challenge to kill cancer cells which grow and multiply over time. More specifically, the poisons in tobacco smoke can damage or change a cell's DNA and once damaged, those cells can begin growing out of control and create a cancer tumor. In addition, vaping has become increasingly popular in recent years among teenagers and young adult populations, and the known addictive nature of nicotine among youth and young adults has developed into a major health concern. These and related issues have become a considerable health risk, including for people who do not smoke or vape but are exposed to the second-hand smoke of another.

Vaping is a process of aerosolizing a solution and inhaling it into the lungs using an electronic cigarette (e-cigarettes) or other vaping device that serves as an alternative to conventional smoking. During the vaping process, e-cigarettes heat a liquid into an aerosol that the user inhales into their lungs, and commonly comprise a replaceable cartridge. A typical e-cigarette cartridge (also referred to as a reservoir or pod) contains four basic ingredients: water; nicotine; flavorings; and, a propylene or vegetable glycerin base commonly referred to as e-liquid. Consumers may choose from a wide variety of vapes and e-cigarette products that contain nicotine, flavorings, and other additives.

Sublingual delivery is most commonly referred to as on of the modes of administering tinctures and systemic drugs, which frequently achieve rapid absorption through the sublingual membrane thereby providing greater bioavailability compared to conventional delivery methods, such as digestion of tablets taken orally (by the mouth).

There are a variety of devices known in the art for vaping. For example, e-cigarettes or “e-cigs,” “cigalikes,” “e-hookahs,” “mods,” “vape pens,” “vapes,” and “tank systems” are vaping devices existing in the art today. By way of example, U.S. Pat. No. 8,511,318 describes an e-cigarette device that is comprised of a cylindrical shell containing an e-cigarette cell, a nicotine solution, a control circuit, and an electro-thermal vaporization nozzle. The device allows a person to suck air through an air suction fitting on one end of the shell, which the patent describes as beneficial as it eliminates tar from the nicotine solution and reduces the risk of fire between inhalations. Another example is provided in U.S. Pat. No. 9,737,093, which describes an e-cigarette device having a separate cartridge and vaporizer unit. The separate cartridge unit comprises a liquid with a seal that keeps the liquid safely within the cartridge tube, but may be pierced by a piercer prior to use. The piercer is described as part of the vaporizer unit, which further comprises a heater and an electronic circuit electrically connected to a battery. Notably, the prior art does not disclose a device for providing an aerosolized and/or micellized liquid nicotine formula. The prior art also fails to disclose an actuator that provides precise dosage such that a known amount of the liquid may be inhaled with each depression/activation. There are other problems with the prior art, including malfunctions with the electronic circuitry, battery failure, mechanical failure, leaking and/or harmful exposure to the liquid or other substance contained by the cartridge, and other problems addressed by the present disclosure.

Although numerous other prior art solutions have attempted to improve upon vaping, sublingual and other delivery devices, none of the solutions effectively provides a pressurized, encapsulated, aerosolized nano mist delivery device such as the device disclosed herein. The prior art also fails to provide a sublingual delivery device that is accurate, reproducible, that provides a known amount of liquid, including a dosage of micellized liquid nicotine formula, that otherwise eliminates the varying dosing and/or delivery issues created by the prior art. A number of other challenges and deficiencies in the art still persist or remained completely unsolved. It is with respect to these and other problems that the present disclosure is contemplated, as will be described in greater detail below.

### SUMMARY

Aspects of the present disclosure relate to an encapsulated, pressurized, sublingual, aerosol nano mist vapor delivery device. The device according to varying embodiments comprises a protective plastic receptacle or housing, which preferably houses at least one canister, and protective plastic receptacle cap. The sublingual delivery device also preferably comprises a custom actuator, metered valve, aluminum canister containing a micellized liquid nicotine formula, a propellant such as, by way of example but not limitation, 1,3,3,3-Tetrafluropropene, HFO 1234ze, Norflurane, HFA 134a, or other acceptable pharmaceutical grade propellant (or a bag-on-valve propellant) allowing a micellized liquid nicotine formula to be expelled from the delivery device as an aerosol nano mist vapor to the sublingual membrane area with the depression of the actuator.

The micellized liquid nicotine formula and novel sublingual delivery device addresses the deficiencies of inaccurate and inconsistent dosing associated with numerous nicotine delivery systems, including e-cigarettes, vapes, vape pens, hookah pens, electronic cigarettes (e-cigarettes or e-cigs), e-cigars, and e-pipes used to describe electronic nicotine delivery systems (ENDS). These and other products that require an “e-liquid” that usually contains nicotine derived from tobacco, as well as flavorings, propylene glycol, vegetable glycerin, and other ingredients, are dangerous and more harmful than the delivery system of the present disclosure.

For example, in these prior art devices, liquid is heated to create an aerosol that the user inhales. The dangers associated with combustible tobacco products like e-cigarettes and the percentage of nicotine and other chemicals inhaled are a concern to the Food and Drug Administration. Robert M. Califf, M. D., FDA commissioner has stated “I'm highly motivated for the FDA to help reduce death and disability caused by these products. We know that there is a demand among adult smokers to use e-cigarette products to try to switch from more harmful combusted cigarettes, but millions of youths are using these products and getting addicted to nicotine.” Further comments by Mitch Zeller, J. D., Director of the FDA's Center for Tobacco Products supports the dangers associated with combustible e-cigarettes, “We are committed to continuing to take the appropriate actions to protect our nation's youth from the dangers of all tobacco products, including e-cigarettes, which remain the most commonly used tobacco product by youth in the United States”.

Through research and experimentation, the Applicant has found that micelles formulated, full spectrum hemp oil (“FSHO”) has demonstrated significantly more bioavailability than non-micelles, unformulated hemp-based oils, and that the FSHO, using micelle technology, is more effective, delivering better than 85% absorption to the body, GI tract and/or skin, with a quicker onset than that of traditional, lower dose cannabidiol-based products. The same distinctions also apply to micellized nicotine oil-based formulas.

The sublingual delivery device preferably is used to deliver a micellized liquid nicotine formula derived from 99.9% pure leaf tobacco, is water-soluble, non-combustible, has no heat, no battery, is smokeless and reduces the nicotine content by as much as eighty percent (80%) compared to traditional cigarettes and electronic nicotine delivery systems (ENDS).

According to one aspect, the novel sublingual delivery device provides a safer (i.e., reduced harm) nicotine delivery device that is non-combustible, has no heating elements, no battery or other power requirements, and is smokeless.

According to yet another aspect, the sublingual delivery device grants an equivalent satisfaction in taste or flavor, and an equivalent nicotine uptake to the user as compared to combustible tobacco products, while at the same time delivering as little as one-fifth the amount of nicotine (an 80% reduction in tobacco/nicotine). The device may thereby lower nicotine levels to minimally addictive or non-addictive levels, preferably by utilizing a micellized liquid nicotine formula. This proprietary method of discovery to micellize nicotine allows for greater bio-absorbable or “bio availability” to the body without changing the molecular form and structure of the nicotine molecule.

Another aspect of the present disclosure relates to an encapsulated sublingual delivery device comprising an actuator, metered valve, protective plastic canister receptacle housing, protective plastic canister receptacle child resistant cap and micellized liquid nicotine formula, which may be introduced into the aluminum canister and expelled as an aerosol, nano mist vapor through the actuator's discharge nozzle by depressing the actuator, thereby delivering a known amount of micellized liquid nicotine formula. The delivery device according to embodiments introduces a known dosage of micellized liquid nicotine formula directly into the sublingual mucosa, thereby affording quick entry into the systemic circulation of the sublingual membrane and providing greater bioavailability to the user.

The micellized liquid nicotine formula is dispensed sublingually with each depression/activation eliminating the varying dosing volumes created in vapes and e-cigarettes. The encapsulated pressurized sublingual aerosol nano mist vapor delivery device eliminates the use of cartridges or reservoirs or pods, the heating element and power source or battery and addresses the deficiencies of inaccurate and inconsistent dosing with vaping by utilizing a microliter metered valve that dispenses a known volume of micellized liquid nicotine formula containing fractionally less nicotine while providing an equivalent feeling of pleasure to the user.

In one aspect, the delivery device comprises a crimped metered valve with a custom actuator attached, which is in communication with the canister receptacle housing. The receptacle housing may further provide a protective child-resistant cap to be placed over the delivery device to protect and secure its position prior to use and provide a child safety feature.

According to yet another specific aspect, the present disclosure relates to a method of delivering a nano mist micellized liquid formula comprising: coupling a metered valve on a canister containing micellized liquid formula; placing the canister and metered valve into a receptacle housing; positioning an actuator above the metered valve; activating the actuator by pressing actuator against the metered valve; piercing a diaphragm on the canister with a valve stem associated with the metered valve; delivering, under pressure, a volume of micellized liquid formula from the canister, through the metered valve, into an expansion chamber; delivering the volume of micellized liquid formula from the expansion chamber through a discharge nozzle.

In another aspect, the propellant is replaced by a bag-on-valve system which delivers an equivalent pressurized metered dose once the micellized liquid nicotine formula is placed inside the bag-on-valve bag and compressed air fills the space between the bag and the aluminum canister allowing the micellized liquid nicotine formula to be dispensed once the actuator is depressed. The micellized liquid nicotine formula is completely separated from the propellant which protects the micellized liquid nicotine formula from any interaction with a pharmaceutical grade propellant.

In one embodiment, at least a portion of the delivery device described herein is reusable. Optionally, a portion of the delivery device is disposable, but in other embodiments the entire delivery device may be reusable or disposable after a set number of uses.

One having skill in the art will appreciate that embodiments of the present disclosure may have various sizes. The sizes of the various devices or their components may be sized based on various factors including, for example, the anatomy of the user, such as the height and weight of the person operating the device, or other physical features of replaceable or reusable components used with the devices described herein, including, for example, width, length and thickness, and the intended dosage(s) provided by the device.

These and other features, utilities, and advantages of the various embodiments of the present invention will be apparent from the following more particular descriptions of the embodiments of the invention as illustrated in the accompanying drawings and defined in the appended claims.

Certain terms are used throughout the following description and claims to refer to a particular system or device components. As one skilled in the art will appreciate, companies may refer to a component by different names. This document does not intend to distinguish between components that differ in name but not function.

The phrases “at least one,” “one or more,” and “and/or,” as used herein, are open-ended expressions that are both conjunctive and disjunctive in operation. For example, each of the expressions “at least one of A, B and C,” “at least one of A, B, or C,” “one or more of A, B, and C,” “one or more of A, B, or C,” and “A, B, and/or C” means A alone, B alone, C alone, A and B together, A and C together, B and C together, or A, B and C together.

Unless otherwise indicated, all numbers expressing quantities, dimensions, conditions, and so forth used in the specification and claims are to be understood as being approximations which may be modified in all instances as required for a particular application of the novel apparatus described herein.

In the following disclosure, the use of “including,” “comprising,” or “having” and variations thereof herein is meant to encompass the items listed thereafter and equivalents thereof as well as additional items. Accordingly, the terms “including,” “comprising,” or “having” and variations thereof can be used interchangeably herein. In the claims, the terms “including” and “comprising” are used in an open-ended fashion, and thus should be interpreted to mean “including, but not limited to . . . .”

The term “a” or “an” entity, as used herein, refers to one or more of that entity. As such, the terms “a” (or “an”), “one or more” and “at least one” can be used interchangeably herein.

The terms “determine,” “calculate,” and “compute,” and variations thereof, as used herein, are used interchangeably and include any type of methodology, process, mathematical operation, or technique.

The term “couple” or “couples” is intended to mean either an indirect or direct connection. When used in a mechanical context, if a first component couples or is coupled to a second component, the connection between the components may be through a direct engagement of the two components, or through an indirect connection that is accomplished via other intermediate components, devices and/or connections. In addition, when used in an electrical context, if a first device couples to a second device, that connection may be through a direct electrical connection, or through an indirect electrical connection via other devices and connections. Connections can occur in a unidirectional, bidirectional or variable directional manner over all known means of network connectivity.

By way of providing additional background, context, and to further satisfy the written description requirements of 35 U.S.C. § 112, the following are incorporated by reference in their entireties for the express purpose of explaining and further describing the various devices and apparatus that may be associated with aerosol and micellized nicotine delivery, in general: U.S. Pat. Nos. 8,511,318; 9,737,093; and, U.S. Pat. No. 11,172,704.

The Summary is neither intended nor should it be construed as being representative of the full extent and scope of the present disclosure. The present disclosure is set forth in various levels of detail in the Summary as well as in the attached drawings and the Detailed Description and no limitation as to the scope of the present disclosure is intended by either the inclusion or non-inclusion of elements, components, etc. in this Summary. Additional aspects of the present disclosure will become more readily apparent from the Detailed Description, particularly when taken together with the drawings.

The above-described benefits, embodiments, and/or characterizations are not necessarily complete or exhaustive, and in particular, as to the patentable subject matter disclosed herein. Other benefits, embodiments, and/or characterizations of the present disclosure are possible utilizing, alone or in combination, as set forth above and/or described in the accompanying figures and/or in the description herein below.

### BRIEF DESCRIPTION OF THE DRAWINGS

Many aspects of the disclosure can be better understood with reference to the following drawings. While several implementations are described in connection with these drawings, the disclosure is not limited to the implementations disclosed herein. On the contrary, the intent is to cover all alternatives, modifications, and equivalents.

In certain instances, details that are not necessary for an understanding of the disclosure or that render other details difficult to perceive may have been omitted. It should be understood, of course, that the disclosure is not necessarily limited to the particular embodiments illustrated in the following Figures. In the drawings:

FIG. 1 is a cross sectional view of a sublingual delivery device for delivering a micellized liquid nicotine formula;

FIG. 2 is a cross sectional view of a metered valve for the device shown in FIG. 1;

FIG. 3 is a cross-sectional view of an actuator assembly for the device shown in FIG. 1;

FIG. 4 is a perspective view of the actuator assembly depicted in FIG. 3;

FIG. 5 is a cross sectional view of a canister for use with the device shown in FIG. 1;

FIG. 6 is a partial elevation view of a receptacle housing for the device shown in FIG. 1;

FIG. 7 is an elevation view of a protective child-resistant cap for use with the device shown in FIG. 1;

FIG. 8 is a side view of the receptacle housing and protective child-resistant cap shown in FIGS. 6-7;

FIG. 9 is an elevation view of the assembled receptacle housing and protective child-resistant cap;

FIG. 10 is a cross-sectional view of an alternate embodiment comprising a bag-on-valve propellant system for use with an aluminum canister;

FIG. 11 is another cross-sectional view of the bag-on-valve system assembled with the aluminum canister of FIG. 10;

FIG. 12 is a cross-sectional view depicting the bag-on-valve propellant system of FIG. 10, wherein the bag-on-valve bag is partially filled with micellized liquid nicotine formula;

FIG. 13 is another cross-sectional view depicting the bag-on-valve propellant system of FIG. 10, wherein the bag-on-valve bag is substantially filled with micellized liquid nicotine formula;

FIG. 14 is a side elevation view of the bag-on-valve propellant system of FIG. 10 with an actuator attached; and

FIG. 15 is a detailed elevation view of the bag-on-valve system of FIG. 10 inserted into a receptacle housing with a child-resistant cap securely attached.

It should be understood that the drawings are not meant to be and/or are not necessarily to scale.

For ease of reference, the following is a listing of components depicted in the drawing figures, some of which are described in greater detail herein:
100 encapsulated pressurized sublingual aerosol nano mist vapor delivery device
102 metered valve
104 valve stem
106 valve internal gasket
108 valve external gasket
110 valve mounting cup
112 valve spring
114 valve metering chamber
116 valve dip tube
118 actuator
120 actuator discharge nozzle
122 actuator base receptor
124 actuator valve sleeve
126 actuator discharge channel
128 actuator ridged finger orientator
130 actuator discharge area
132 aluminum canister
134 aluminum canister lip
136 aluminum canister throat
138 aluminum canister rim
140 aluminum canister base
142 aluminum canister concave base bottom
144 protective plastic canister receptacle housing
146 plastic base receptacle rim
148 plastic base receptacle child resistant cap channel
150 plastic base receptacle recessed lip
152 protective plastic canister base
154 protective plastic canister receptacle child resistant cap
156 protective plastic canister receptacle child resistant cap base
158 plastic child resistant rib
160 liquid propellant
162 micellized liquid formula
164 air pocket
166 bag-on-valve system
168 bag-on-valve metered valve
170 bag-on-valve bag
172 compressed air propellant
174 bag-on-valve inflation bag
176 encapsulated pressurized sublingual aerosol nano mist delivery device having a bag on valve system

### DETAILED DESCRIPTION

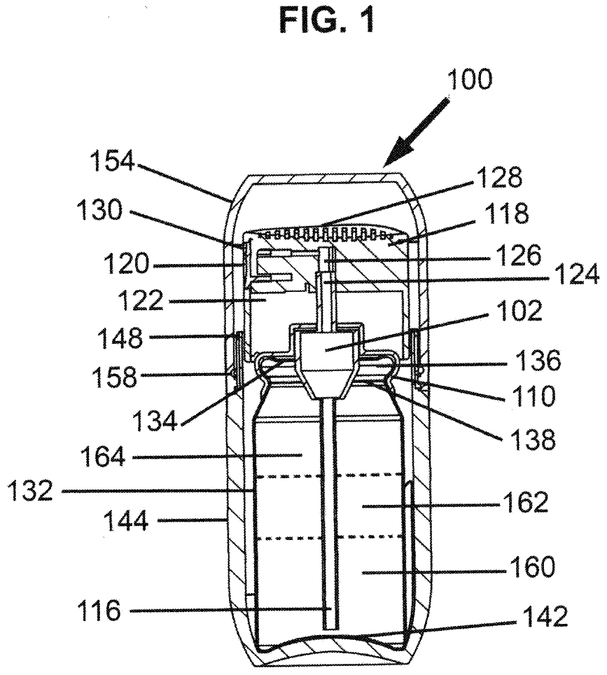

The following disclosure is directed to various embodiments, including those depicted in FIGS. 1-15 appended hereto. As depicted in FIG. 1, the pressurized sublingual delivery device eliminates the use of a power source or battery, which are commonly employed in prior art devices and have been known to explode without warning and cause serious injuries including burns, blindness or in extreme cases, death.

Embodiments described herein relate to an encapsulated, pressurized, sublingual aerosol nano mist vapor delivery device that delivers a precise dose of micellized liquid nicotine formula. According to embodiments, the precise or controlled dose is achieved with each depression of an actuator, which preferably is positioned on top of a metered valve crimped to a pressurized aluminum canister. The aluminum canister may be Fluorinated Ethylene Propylene (FEP) coated, so as to avoid any undesired chemical interaction between the micellized liquid formula and the aluminum canister.

In embodiments, the micellized liquid formula may be comprised of at least one of a micellized liquid nicotine, micellized liquid cannabidiol, micellized liquid tetrahydrocannabinol, and a micellized nicotine salt.

The encapsulated pressurized sublingual aerosol nano mist vapor delivery device is created by placing an aluminum canister, containing a micellized liquid nicotine formula having a bag-on-valve system or pharmaceutical grade liquid propellant residing inside and crimped metered valve attached with actuator positioned on top, into the protective plastic canister receptacle housing with a protective child resistant protective plastic cap secured on top.

The encapsulated pressurized sublingual delivery device preferably comprises a metered valve crimped onto the aluminum canister, such as by use of a metal collette, which crimps the metered valve mounting cup of the aluminum metered valve against the rim of the aluminum canister. The crimp securely holds the metered valve onto the aluminum canister which once gassed maintains approximately 70 pounds per square inch (psi) pressure in the aluminum canister.

The actuator, preferably made from high quality plastic resins, has a discharge nozzle that may be altered to expel various nano mist or micron-sized vapor particles while delivering a precise volume of micellized liquid nicotine formula to the sublingual membrane area once depressed. The volume of micellized formulation delivered with each activation of the actuator is preferably within the range of 50 to 100 microliters. In one embodiment, the volume is precisely controlled at 100 microliters by utilizing a metered valve configured to release 100 microliters. In another embodiment, the volume is precisely controlled at 65 microliters.

According to one particular aspect, the method of delivering a known volume of micellized liquid nicotine formula is as follows: once the valve stem has been fully depressed, a valve stem side piercing passes through a diaphragm. The volume of micellized liquid nicotine formula enters into the metering chamber, which includes a precise, controlled volume of micellized liquid nicotine formula and propellant based on the configuration of metered valve. From the first actuation until the last actuation, the pressure in the canister remains constant allowing a controlled, consistent and precise dose of micellized liquid formula and propellant to be expelled through the actuator's discharge nozzle.

Referring now in detail to FIG. 1, various components of the pressurized, encapsulated, sublingual delivery device 100 are illustrated. According to a preferred embodiment, the delivery device 100 comprises an aluminum canister 132 having a generally concave base bottom 142 positioned securely inside protective plastic canister receptacle housing 144 with a protective plastic canister receptacle child resistant cap 154. The child resistant cap 154 may be attached by pushing plastic ribs 158 into the plastic base receptacle channels 148. Mounting cup 110 of metered valve 102 is crimped over aluminum canister lip 134, aluminum canister throat 136 and against aluminum canister rim 138 of aluminum canister 132, thereby allowing a pressurized liquid propellant such as, 1,3,3,3-Tetrafluropropene, HFO 1234ze, Norflurane, HFA 134a or similar pharmaceutical grade propellant 160 to be introduced under pressure into aluminum canister 132.

The volume of liquid propellant 160 is pre-determined by the volume of micellized liquid nicotine formula 162 introduced into the aluminum canister 132. It is known by those skilled in the art that pressurized liquid such as 1,3,3,3-Tetrafluropropene, HFO 1234ze, Norflurane, HFA 134a or similar pharmaceutical grade propellant 160 introduced into aluminum canister 132 containing a micellized liquid nicotine formula 162 will expand as outside temperatures increase. The pre-determined volume of liquid propellant 160 and micellized liquid nicotine formula 162 introduced into canister 132 are adjusted to allow expansion when temperatures and pressure increase inside aluminum canister 132.

In a preferred embodiment, the starting pressure of the prefilled aluminum canister 132 is approximately 70 psi, (pounds per square inch), at an outside temperature of approximately 72 degrees Fahrenheit or 22.2 Celsius. Once micellized liquid nicotine formula 162 and liquid propellant 160 are introduced into aluminum canister 132, air pocket 164 is required to allow expansion inside aluminum canister 132 once outside temperatures increase beyond 72 degrees Fahrenheit or 22.2 Celsius. Actuator 118, which preferably resides over metered valve sleeve 124 via the actuator base receptor 122 is activated by depressing actuator ridged finger control 128. A micellized liquid formula 162 and liquid propellant 160 under pressure are drawn up through valve dip tube 116 and forced through actuator discharge channel 126 and out actuator discharge nozzle 120 with a uniform nano mist pattern created from actuator discharge area 130.

It has been discovered that micellized liquid nicotine formula 162 when combined with liquid propellant Norflurane, HFA 134a remains miscible forming a homogeneous mixture without separation between combined liquid propellant 160 and micellized liquid nicotine formula 162. This discovery simplifies the use of the encapsulated sublingual aerosol nano mist vapor delivery device 100 as it is not required to be shaken to mix the liquid propellant 160 and micellized liquid nicotine formula 162 together prior to use. Without micellization pure nicotine would remain in suspension and could cause health issues to the user. When liquid propellant 160 and micellized liquid formula 162 are introduced into a clear glass vial, having metered valve 102 attached, both liquid solutions remain miscible, visibly clear, with a viscosity similar to that of water. This revelation and discovery will challenge all combustible tobacco products as it allows for a safer, easy to use, quicker uptake with increased bioavailability into the sublingual mucosa while decreasing nicotine levels up to eighty percent (80%).

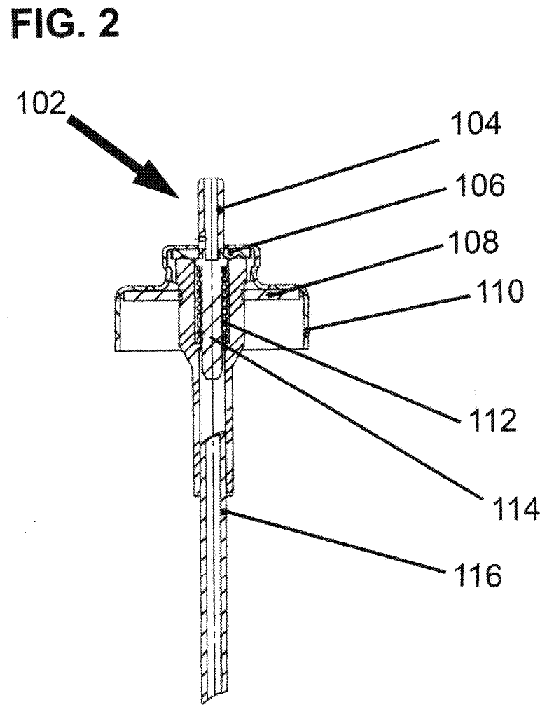

FIG. 2 is a cross sectional view of metered valve 102 comprised of metered valve stem 104, metered valve internal gasket 106, metered valve external gasket 108, metered valve mounting cup 110, metered valve spring 112, metered valve metering chamber 114, and metered valve dip tube 116. Once micellized liquid nicotine formula 162 (not shown) and liquid propellant 160 (not shown) are introduced into aluminum canister 132, a combined pressurized homogeneous water-soluble liquid solution is created and forced into metered valve dip tube 116 and metering chamber 114. Once actuator 118 (not shown) is depressed metered valve stem 104 is pushed downward past metered valve internal gasket 106 and metered valve external gasket 108 overcoming resistance from metered valve spring 112 allowing micellized liquid formula 162 (not shown) and liquid propellant 160 (not shown) to be expelled through actuator discharge nozzle 120 (not shown).

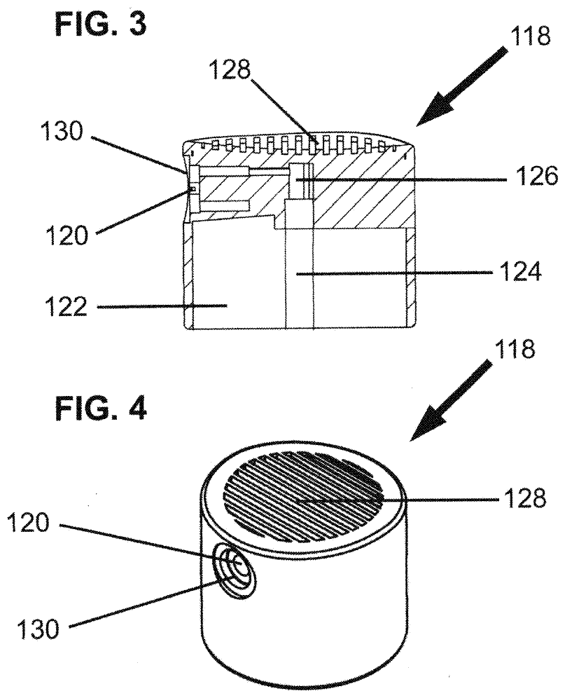

FIG. 3 is a cross-sectional side view of actuator 118, having an actuator base receptor 122 securely placed over metered valve stem 104 (not shown) via actuator valve sleeve 124. Once actuator 118 is depressed by pushing downward on actuator ridged finger control 128, micellized liquid nicotine formula 162 (not shown) and liquid propellant 160 (not shown) under pressure are forced through actuator discharge channel 126 and actuator discharge nozzle 120 creating a micellized nicotine nano mist vapor spray pattern from actuator discharge area 130. Discharge nozzle 120 can be altered in diameter to provide various nano mist, vapor micron particle sizes.

FIG. 4 is a perspective view of actuator 118 showing actuator ridged finger control 128 which is concave and ridged to accommodate finger location and correct direction of micellized nicotine nano mist spray. The actuator discharge area 130 is circular allowing a micellized nicotine nano mist spray to be expelled uniformly from the actuator discharge nozzle 120.

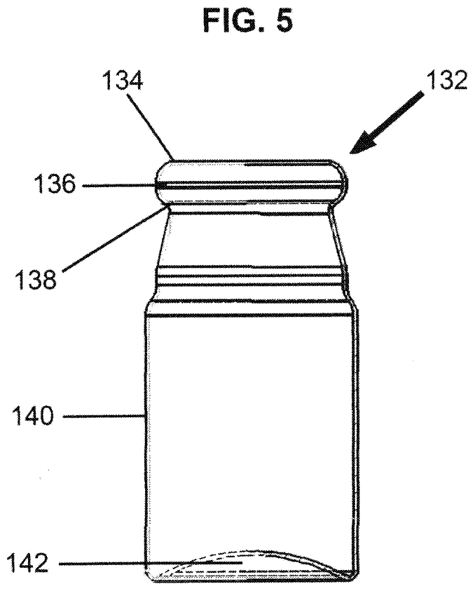

FIG. 5 is a cross-sectional view of aluminum canister 132, having an aluminum canister concave base bottom 142 and aluminum canister base 140 where metered valve 122 (not shown) resides over aluminum canister lip 134 and aluminum canister throat 136. When metered valve 122 (not shown) is crimped to aluminum canister 132, metered valve mounting cup 110 (not shown) is crimped firmly against aluminum canister rim 138 allowing a secure fit and safe pressure to remain in aluminum canister 132.

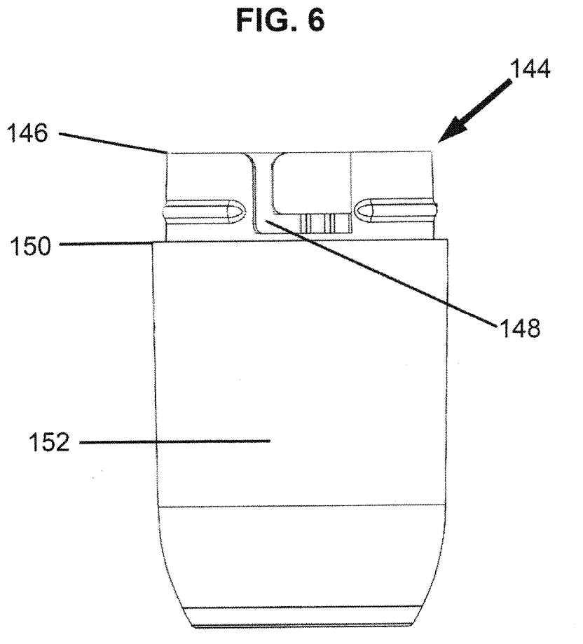

FIG. 6 is a partial elevation view of protective plastic canister receptacle housing 144, comprising a protective plastic canister base 152, plastic base receptacle recessed lip 150, plastic base receptacle child resistant cap channels 148 and plastic base receptacle rim 146.

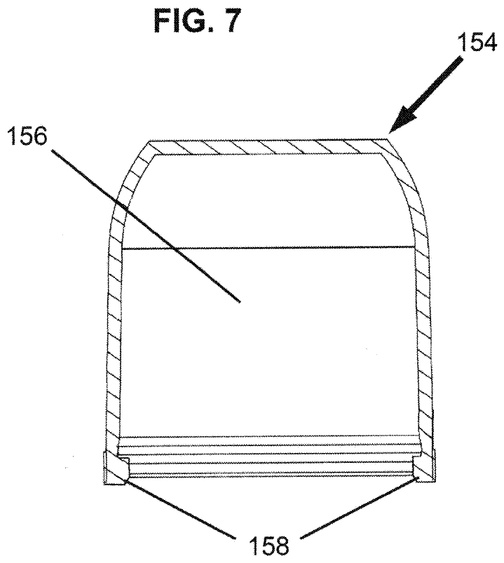

FIG. 7 is an elevation view of protective plastic canister receptacle child resistant cap 154 having protective plastic canister receptacle child resistant cap base 156 and plastic child resistant ribs 158.

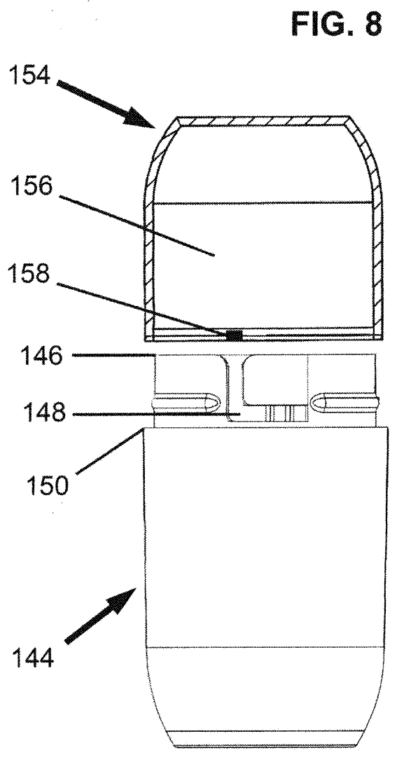

FIG. 8 is a side view of protective plastic canister receptacle child resistant cap 154 residing over the receptacle housing 144. Once plastic child resistant ribs 158, located on both sides of protective plastic canister receptacle child resistant cap 154, are aligned above plastic base receptacle child resistant cap channels 148, located on both sides of receptacle housing 144, pushing downward over plastic base receptacle rim 146 and against plastic base receptacle recessed lip 150 and turning counterclockwise through plastic base receptacle child resistant cap channels 148 locks the protective plastic canister receptacle child resistant cap 154 together with protective plastic canister receptacle housing 144.

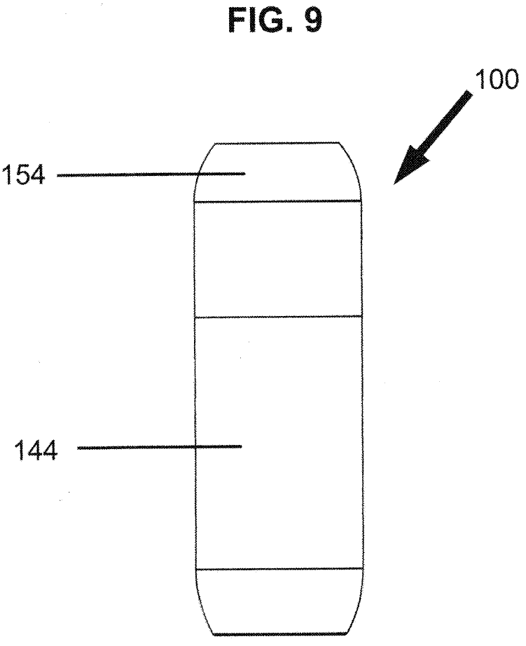

FIG. 9 is a cross-sectional view of encapsulated pressurized sublingual aerosol nano mist vapor delivery device 100 showing protective plastic canister receptacle child resistant cap 154 attached to protective plastic canister receptacle housing 144. Aluminum canister 132 containing micellized liquid nicotine formula 162 and liquid propellant 160 with metered valve 102 crimped onto aluminum canister 132 and actuator 118 positioned on metered valve 102 are not shown.

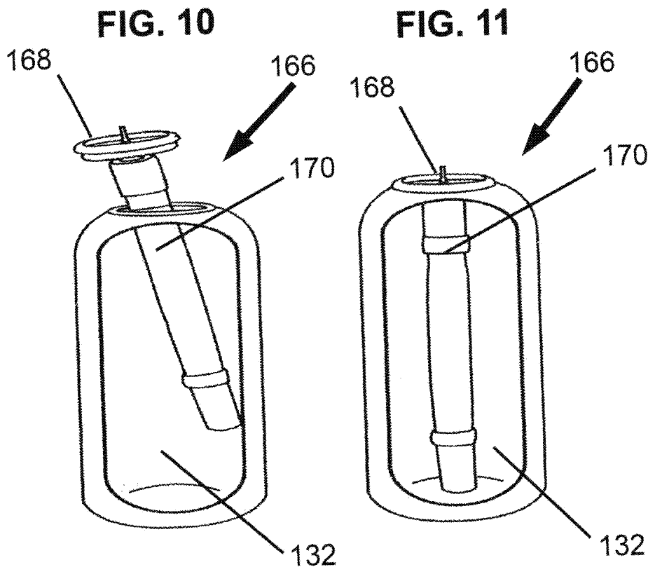

FIGS. 10-11 are cross-sectional views of bag-on-valve system 166 depicting insertion of bag-on-valve 170, having bag-on-valve metered valve 168 attached, into aluminum canister 132.

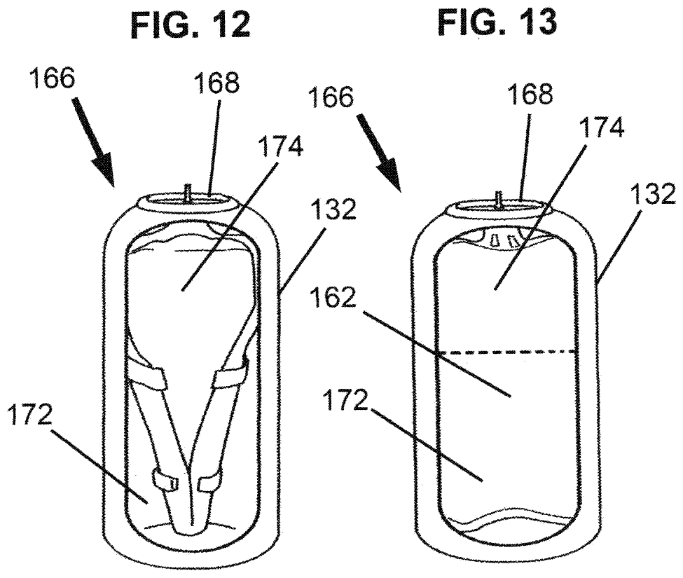

FIGS. 12-13 are cross-sectional views of a bag-on-valve system 166 depicting the bag-on-valve metered valve 168 crimped onto aluminum canister 132, filling aluminum canister 132 with compressed air propellant 172, and then filling the bag-on-valve bag 174 with micellized liquid nicotine formula 162.

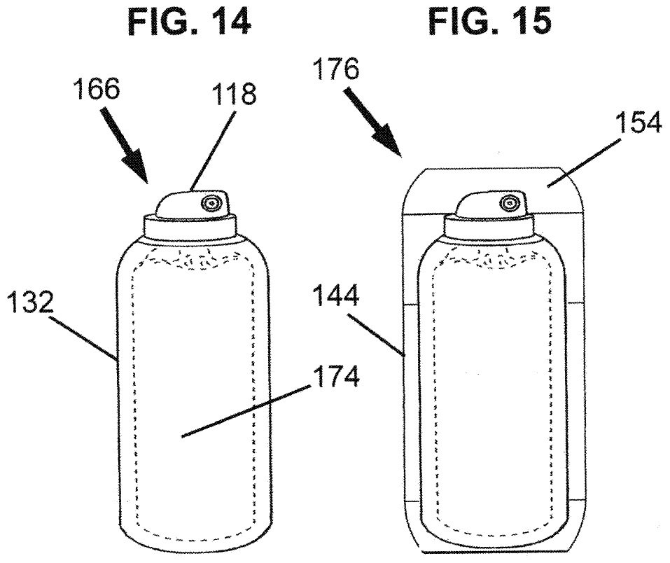

FIG. 14 is a side view of the bag-on-valve system 166 with an actuator 118 placed over bag-on-valve metered valve 168 (not shown) crimped onto a canister 132. Micellized liquid nicotine formula 162 (not shown) resides inside bag-on-valve inflated bag 174.

FIG. 15 is a detailed elevation view of the sublingual delivery device 176 with insertion of bag-on-valve system 166 into the protective plastic canister receptacle housing 144, further comprising a protective child-resistant cap 154 secured in place.

FIGS. 1-15 may depict more than all of the components required to operate the device. It is also understood that the device design may comprise additional surface features in certain embodiments than those shown in FIGS. 1-15, including surface texturing, coloring, packaging, ergonomic shape, contouring, sheathing, etc. Thus, the design of FIGS. 1-15 is exemplary and non-limiting as to the scope of the present disclosure.

With respect to the embodiments described above, it is expressly understood that such embodiments may be incorporated for use in practicing the novel methods described herein. In certain embodiments, those methods may comprise greater or fewer steps than as described above. By way of example, but not limitation, one step for use with the various embodiments described above may comprise the step of removing and replacing the canister or securing a safety guard, with different steps included in the method of operation provided accordingly. Thus, variations on this and other steps, and the inclusion or exclusion of additional steps described herein, are expressly contemplated by the present disclosure.

It is expressly understood that where the term “consumer” has been used to describe the various embodiments of the disclosure, the term should not be construed as limiting in any way. For instance, a consumer could be a human user or, in a veterinary application, an animal. Therefore, it is intended that the device and methods described herein apply equally to veterinary science as they would if practiced on a human being. The apparatus and methods described herein therefore have application beyond controlled dosage of nicotine, by way of example.

The foregoing discussion of the disclosure has been presented for purposes of illustration and description. The foregoing is not intended to limit the disclosure to the form or forms disclosed herein. In the foregoing Detailed Description for example, various features of the disclosure are grouped together in one or more embodiments for the purpose of streamlining the disclosure. This method of disclosure is not to be interpreted as reflecting an intention that the claimed disclosure requires more features than are expressly recited in each claim. Rather, as the following claims reflect, inventive aspects lie in less than all features of a single foregoing disclosed embodiment. Thus, the following claims are hereby incorporated into this Detailed Description, with each claim standing on its own as a separate preferred embodiment of the disclosure.

The present inventions, in various embodiments, include components, methods, processes, systems and/or apparatuses substantially as depicted and described herein, including various embodiments, subcombinations, and subsets thereof. Those of skill in the art will understand how to make and use the present inventions after understanding the present disclosure. The present inventions, in various embodiments, include providing devices and processes in the absence of items not depicted and/or described herein or in various embodiments hereof, including in the absence of such items as may have been used in previous devices or processes, e.g., for improving performance, achieving ease and\or reducing cost of implementation.

Moreover, though the present disclosure has included description of one or more embodiments and certain variations and modifications, other variations and modifications are within the scope of the disclosure, e.g., as may be within the skill and knowledge of those in the art, after understanding the present disclosure. For further illustration, the information and materials supplied with the provisional application from which this application claims priority is expressly made a part of this disclosure and incorporated by reference herein in their entirety. This form of disclosure, therefore, is intended to obtain rights which include alternative embodiments to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

What is claimed is:

1. A sublingual delivery device comprising:
a protective receptacle housing;
a protective receptacle cap configured to be placed over the protective receptacle housing;
a canister received within the protective receptacle housing;
a metered valve crimped to at least a portion of the canister and received within the protective receptacle housing;
an actuator positioned over the metered valve, the actuator configured to deliver, sublingually, at least one micellized liquid formula.

2. The sublingual delivery device of claim 1 further comprising a childproof safety guard that fits over the actuator.

3. The sublingual delivery device of claim 1, wherein the at least one micellized liquid formula comprises a micellized liquid nicotine formula.

4. The sublingual delivery device of claim 1, wherein the at least one micellized liquid formula comprises a micellized liquid cannabidiol formula.

5. The sublingual delivery device of claim 1, wherein the at least one micellized liquid formula comprises a micellized liquid tetrahydrocannabinol formula.

6. The sublingual delivery device of claim 1, wherein a predetermined volume of the at least one micellized liquid formula is delivered through the actuator discharge with each activation of the actuator.

7. The sublingual delivery device of claim 6, wherein the predetermined volume is in the range of 50 to 100 microliters.

8. The sublingual delivery device of claim 6, wherein the predetermined volume is 100 microliters.

9. The sublingual delivery device of claim 6, wherein the predetermined volume is 65 microliters.

10. The sublingual delivery device of claim 6, wherein the at least one micellized liquid formula comprises at least a nicotine salt.

11. A method of delivering a micellized liquid formula directly into the sublingual mucosa of a user, comprising:
coupling a metered valve on a canister containing a micellized liquid formula;
placing the canister and metered valve into a receptacle housing;
positioning an actuator above the metered valve;
activation the actuator by pressing actuator against the metered valve;
piercing a diaphragm on the canister with a valve stem associated with the metered valve;
delivering a volume of micellized liquid formula from the canister, through the metered valve, into an expansion chamber;
delivering the volume of micellized liquid formula from the expansion chamber through a discharge nozzle for providing the volume of micellized liquid formula directly to the sublingual mucosa of the user.

12. The method of claim 11, wherein the delivery step comprises delivering at least one of a micellized liquid nicotine, micellized liquid cannabidiol, micellized liquid tetrahydrocannabinol, and a micellized nicotine salt.

13. The method of claim 11, wherein the method further comprises placing a safety guard about the actuator to prevent further activation.

14. The method of claim 11, wherein the volume of micellized liquid formula is 100 microliters.

15. The method of claim 11, wherein the volume of micellized liquid formula is 65 microliters.

16. The method of claim 11, wherein the volume of micellized liquid formula is in the range of 50 to 100 microliters.

17. The sublingual delivery device of claim 1 further comprising a bag-on-valve system.

18. The method of claim 11 further comprising a bag-on-valve system.
